(19) United States
(12) Patent Application Publication
(10) Pub. No.: US 2007/0092524 A1
(43) Pub. Date: Apr. 26, 2007

(54) Method for the treatment and prophylaxis of avian influenza infection

(76) Inventors: Jacob Nusbacher, Ra'anana (IL); Vered Caplan, Kiryat Ono (IL)

Correspondence Address: PEARL COHEN ZEDEK LATZER, LLP, 1500 BROADWAY 12TH FLOOR, NEW YORK, NY, 10036, US

(21) Appl. No.: 11/584,568

(22) Filed: Oct. 23, 2006

Related U.S. Application Data
(60) Provisional application No. 60/728,764, filed on Oct. 21, 2005.
(60) Provisional application No. 60/729,196, filed on Oct. 24, 2005.

(51) Int. Cl. A61K 39/42

(52) U.S. Cl. 424/159.1

(57) ABSTRACT

This invention relates to methods and compositions for treatment and prevention of Avian Influenza. Specifically, the invention relates to the use of immunoglobulins obtained from a subject immune to Avian Influenza in the preparation and use of pharmaceutical preparations for the treatment of Avian Influenza.

### CROSS REFERENCE TO RELATED APPLICATIONS

This application is a US Application claiming priority from U.S. Provisional Patent Application No. 60/728,764, filed 21 Oct. 2005 and U.S. Provisional Patent Application No. 60/729,196, filed 24 Oct. 2005, both which are hereby incorporated by reference in their entirety

### FIELD OF INVENTION

This invention is directed to methods and compositions for treatment and prevention of Avian Influenza. Specifically, the invention relates to the use of immunoglobulins obtained from a subject immune to Avian Influenza in the preparation and use of pharmaceutical formulations for the treatment of Avian Influenza.

### BACKGROUND OF THE INVENTION

Avian influenza A viruses do not generally infect humans. Nevertheless, there have been a number of human cases reported since 1997. Most cases of avain influenza infection in humans are thought to have resulted from direct contact with infected poultry. Human-to-human transmission has also occurred, but to date has not been widely sustained in the human population. Nevertheless, this potential exists. Since 1997, a number of avian influenza viruses has been identified that have infected humans. These include the following:

H5N1, Hong Kong, Special Administrative Region, 1997: Highly pathogenic avian influenza A (H5N1, referring to different combinations of the viral envelope glycoproteins haemagglutinin [H] and neuraminidase [N]), infections occurred in both poultry and humans. This was the first time an avian influenza A virus transmission directly from birds to humans had been found. During this outbreak, 18 people were hospitalized and six of them died. To control the outbreak, authorities killed about 1.5 million birds to remove the source of the virus. Scientists determined that the virus spread primarily from birds to humans, though rare person-to-person infection was noted. Studies at the genetic level further determined that the virus had been transmitted directly from birds to humans.

H9N2, China and Hong Kong, Special Administrative Region, 1999: Low pathogenic avian influenza A (H9N2) virus infection was confirmed in two children and resulted in uncomplicated influenza-like illness. Both subjects recovered, and no additional cases were confirmed. The source is unknown, but the evidence suggested that poultry was the source of infection and the main mode of transmission was from bird to human. However, the possibility of person-to-person transmission could not be ruled out. Several additional human H9N2 infections were reported from China in 1998-99.

H7N2, Virginia, 2002: Following an outbreak of H7N2 among poultry in the Shenandoah Valley poultry production area, one person was found to have serologic evidence of infection with H7N2.

H5N1, China and Hong Kong, Special Administrative Region, 2003: Two cases of highly pathogenic avian influenza A (H5N1) infection occurred among members of a Hong Kong family that had traveled to China. One person recovered, the other died. How or where these two family members were infected was not determined. Another family member died of a respiratory illness in China, but no testing was done.

H7N7, Netherlands, 2003: The Netherlands reported outbreaks of influenza A (H7N7) in poultry on several farms. Later, infections were reported among pigs and humans. In total, 89 people were confirmed to have H7N7 influenza virus infection associated with this poultry outbreak. These cases occurred mostly among poultry workers. H7N7-associated illness included 78 cases of conjunctivitis (eye infections); 5 cases of conjunctivitis and influenza-like illnesses with cough, fever, and muscle aches only; 2 cases of influenza-like illness only; and 4 cases that were classified as “other.” There was one death among the 89 total cases. It occurred in a veterinarian who visited one of the affected farms and developed acute respiratory distress syndrome and complications related to H7N7 infection. The majority of these cases occurred as a result of direct contact with infected poultry; however, Dutch authorities reported three possible instances of transmission from poultry workers to family members. Since then, no other instances of H7N7 infection among humans have been reported.

H9N2, Hong Kong, Special Administrative Region, 2003: Low pathogenic avian influenza A (H9N2) infection was confirmed in a child in Hong Kong. The child was hospitalized and recovered.

H7N2, New York, 2003: In November 2003, a subject with serious underlying medical conditions was admitted to a hospital in New York with respiratory symptoms. One of the initial laboratory tests identified an influenza A virus that was thought to be H1N1. The subject recovered and went home after a few weeks. Subsequent confirmatory tests conducted in March 2004 showed that the subject had been infected with avian influenza A (H7N2) virus.

H7N3 in Canada, 2004: In February 2004, human infections of highly pathogenic avian influenza A (H7N3) among poultry workers were associated with an H7N3 outbreak among poultry. The H7N3-associated, mild illnesses consisted of eye infections. H5N1, Thailand and Vietnam, 2004, and other outbreaks in Asia during 2004 and 2005: In January 2004, outbreaks of highly pathogenic influenza A (H5N1) in Asia were first reported by the World Health Organization.

From these reports, it is clear that human avian influenza infection has occurred in Asia, North America and Europe. Nevrtheless, infection with the highly virulent H5N1 strain seems to have occurred predominantly in Asia.

Symptoms of avian influenza infection range from typical influenza type symptoms—fever, cough, sore throat and muscle aches—to conjunctivitis, pneumonia, acute respiratory distress, and other lfe-threatening complications.

Based on recorded patterns, influenza pandemics can be expected to occur, on average, three to four times each century when new virus subtypes emerge and are readily transmitted from person to person. However, the occurrence of influenza pandemics is unpredictable. In the 20th century, the influenza pandemic of 1918-1919, caused an estimated 40 to 50 million deaths worldwide and was followed by pandemics in 1957-1958 and 1968-1969. Most experts now agree that another influenza pandemic is inevitable and possibly imminent. With the potential outbreak of avian influenza and it's constantly changing virus, there is evidently a need for an effective treatment or vaccine.

Antiviral drugs, some of which can be used for both treatment and prevention, are clinically effective against influenza A virus strains in otherwise healthy adults and children, but are also expensive and supplies are limited.

### SUMMARY OF THE INVENTION

In one embodiment, the invention provides a composition for preventing Avian Influenza in a subject, comprising immunoglobulins, fragments thereof, Avian Influenza antibodies, or combinations thereof, wherein said immunoglobulins or their fragments Avian Influenza antibodies, or combinations thereof, are obtained from plasma of one or more subjects immune to said Avian Influenza.

In another embodiment, the invention provides a method of preventing or treating Avian Influenza in a subject, comprising administering to said subject a composition comprising immunoglobulins, fragments thereof, Avian Influenza antibodies, or combinations thereof, wherein said immunoglobulins, fragments thereof, Avian Influenza antibodies, or combinations thereof, are obtained from plasma of one or more subjects immune to said Avian Influenza.

In one embodiment, the invention provides a method of producing a pharmaceutical preparation for the prevention or treatment of Avian Influenza, comprising: obtaining plasma from a one or more subjects immune to Avian Influenza; pooling said plasma; fractionating said plasma, wherein said fractionation isolates or purifies immunoglobulins, fragments thereof, Avian Influenza antibodies, or a combination thereof from the plasma; and concentrating said immunoglobulins, fragments thereof, Avian Influenza antibodies, or combinations thereof.

### DETAILED DESCRIPTION OF THE INVENTION

Avian Influenza is an infectious disease of birds caused by type A strains of the influenza virus. All birds are susceptible to infection with Avian Influenza, though some bird species are more resistant to infection than others. Infection causes a wide spectrum of symptoms in birds, ranging from mild illness to a highly contagious and rapidly fatal disease resulting in severe epidemics. The latter is known as “highly pathogenic Avian Influenza”; this form is characterized by a sudden onset, severe illness, and rapid death, with bird mortality rate in birds, that can approach 100%. Fifteen subtypes of influenza viruses are known to infect birds, providing a vast pool of viruses potentially circulating in bird populations. To date, all outbreaks of the highly pathogenic form of the virus in birds, have been caused by influenza A viruses of subtypes H5 and H7.

All type A influenza viruses, including those that regularly cause seasonal influenza epidemics in humans, are genetically labile and unstable. Influenza viruses lack mechanisms for the “proofreading” and repair of errors that occur during viral replication. As a result of these uncorrected errors, the genetic composition of the viruses changes as they replicate in humans; and the existing strain is replaced with a new antigenic variant. These constant, permanent and usually small changes in the antigenic composition of influenza A viruses are known in one embodiment as “antigenic drift”. In addition, influenza A viruses, including subtypes from different species, can swap or “reassort” genetic materials and merge. This reassortment process, known in one embodiment as “antigenic shift”, results in a novel subtype different from both parent viruses. As populations will have no immunity to the new subtype, and as no existing vaccines can confer protection, antigenic shift has historically resulted in highly lethal pandemics. For a pandemic to happen, the novel influanza subtype needs to have genes from human influenza viruses that make it readily transmissible from person to person for a sustainable period and have the virulence to cause the pathologic changes and the clinical symptoms in humans

The term “antigenic drift” refers in one embodiment to the accumulation of point mutations in the antigenic domain of the hemagglutinin A (HA) protein of the virus. In another embodiment, due to antigenic drift, viruses with a changed antigenic structure emerge. Said changed antigenic structure will not be recognized by the host's acquired immunity, regardless of whether this immunity is acquired by natural infection or vaccination.

In one embodiment, a single virion must contain each of the eight unique Infuenza A RNA segments to be infectious. In another embodiment, the incorporation of RNAs into virions is random. In one embodiment, the term “reassortment” refers to the random incorporation of RNA segments allowing the generation of progeny viruses containing novel combinations of genes wherein cells are infected with different parent viruses. In another embodiment, the progeny virion can be the result of double reassortment, such as the initial North Carolina H3N2 isolate that contained gene segments similar to those of the human (HA, NA, and PB1) and classic swine (NS, NP, M, PB2, and PA) lineages. In one embodiment, the progeny virion can be the result of triple reassortment, such as the H3N2 virus isolate circulating currently in the U.S. swine population, containing avian-like (PA and PB2), swine-like (M, NP, and NS), and human-like (HA, NA, and PB1) gene segments. In one embodiment, antibodies collected from subjects exposed to a triple assortant virus, or in another embodiment, a double assortant virus are used in the compositions described herein. In one embodiment, antibodies collected from subjects exposed to a higher than triple assortant virus are used in the compositions described herein.

Pigs have been shown to be susceptible to infection with both avian and mammalian viruses, including human strains, therefore they can serve as a reactor for the scrambling of genetic material from human and avian viruses, resulting in the emergence of a novel subtype. A second possible mechanism is that, for at least some of the 15 Avian Influenza virus subtypes circulating in bird populations, humans themselves can serve as the reactor for antigenic shift.

Subjects who are infected with the Avian Influenza virus and recover, mount, or will have mounted, an immune response to this virus and make IgG or IgM antibodies against the virus. In one embodiment, these individuals are immune to the Avian Influenza virus. As a result, their plasma is used in another embodiment as a therapeutic agent to prevent Avian Influenza infection in individuals who are not immune, or as treatment in those subjects who are ill with the disease. In one embodiment, the plasma of immune individuals with immunity to Avian Influenza is processed to manufacture an immunoglobulin preparation which is effective in preventing and/or treating Avian Influenza.infection.

According to this aspect of the invention and in one embodiment, the invention provides a composition for preventing Avian Influenza in a subject, comprising immunoglobulins, fragments thereof, Avian Influenza antibodies, or combinations thereof, wherein said immunoglobulins or their fragments, Avian Influenza antibodies, or combinations thereof are obtained from plasma of one or more subjects immune to said Avian Influenza.

In one embodiment, said composition further comprises an additional therapeutic agent, a vaccine, an adjuvant or a combination thereof.

Adjuvants suitable for use in the compositions and methods described herein include, but are not limited to several adjuvant classes such as; mineral salts, e.g., Alum, aluminum hydroxide, aluminum phosphate and calcium phosphate; surface-active agents and microparticles, e.g., nonionic block polymer surfactants (e.g., cholesterol), virosomes, saponins (e.g., Quil A, QS-21, Alum and GPI-0100), proteosomes, immune stimulating complexes, cochleates, quarterinary amines (dimethyl diocatadecyl ammonium bromide (DDA)), pyridine, vitamin A, vitamin E; bacterial products such as the RIBI adjuvant system (Ribi Inc.), cell wall skeleton of Mycobacterum phlei (Detox.®.), muramyl dipeptides (MDP) and tripeptides (MTP), monophosphoryl lipid A, Bacillus Calmete-Guerin (BCG), heat labile E. coli enterotoxins, cholera toxin, trehalose dimycolate, CpG oligodeoxnucleotides; cytokines and hormones, e.g., interleukins (IL-1, IL-2, IL-6, IL-12, IL-15, IL-18), granulocyte-macrophage colony stimulating factor, dehydroepiandrosterone, 1,25-dihydroxy vitamin D3; polyanions, e.g., dextran; polyacrylics (e.g., polymethylmethacrylate, Carbopol 934P); carriers e.g., tetanus toxid, diptheria toxoid, cholera toxin B subnuit, mutant heat labile enterotoxin of enterotoxigenic E. coli (rmLT), heat shock proteins; oil-in-water emulsions e.g., AMPHIGEN.RTM. (Hydronics, USA); and water-in-oil emulsions such as, e.g., Freund's complete and incomplete adjuvants.

Two forms of immunization have been utilized with great success for more than 50 years both for the treatment and prevention of bacterial and viral infections. These are termed active and passive immunization.

In one embodiment, active immunization (also called vaccination) involves the administration of either a live, attenuated or killed microorganism, or a portion of said microorganism in order “prime” the cellular immune system and to elicit an antibody response in the subject. Microoganisms may be a baterium, a virus, a virus-like particle or a combination therof. The antibody response—which results in certain embodiments, is the ability of the subject's immune system to select, synthesize and secrete antibodies that will kill the specific invading microorganism—takes some weeks or months to occur, during which time the subject remains vulnerable to the microorganism. However, once vaccinated, the subject retains the ability to defend himself against that microorganism for part or the rest of his or her life, at least in part by raising specific antibodies against the microorganism when exposed. (although booster immunizations may be required periodically). Active immunization has been shown to be highly effective in conferring long-term protection against certain conditions and is generally administered when the subject is well and has not been recently exposed to the innoculum. Examples of active viral vaccines include smallpox, polio, and hepatitis B.

Passive immunization involves in another embodiment, the administration to the subject of a purified immunoglobulin preparation which contains relatively high quantities of one or more antibodies specific to the target microorganism. In one embodiment, passive administration of such antibodies confers immediate but temporary immunity against a specific microorganism, usually for the time that the antibodies are present in the body (perhaps a month or two). As a result, passive immunization is used when the subject has been recently exposed to a specific microorganism or is at high risk of being exposed to a microorganism in an attempt to prevent, or modify the severity of, disease caused by the microorganism in question. Examples of viral passive antibodies given prophylactically include Rabies immuneglobulin and Varicella-Zoster immuneglobulin. In some cases, passive immunization is given when the subject is already ill, as a therapeutic agent. Examples of passive immunization include but are not limited to viral antibodies given therapeutically, include Hepatitis B immuneglobulin [in liver transplants for Hepatitis B liver failure] and Cytomegalovirus immuneglobulin. These therapies have proven to be highly effective as well.

The efficacy of all immunization programs for the prevention and treatment of bacterial or viral infections is based in one embodiment, on the magnitude of circulating antibody levels. Dosing schedules and product specifications are constructed in certain embodiments around the level of antibodies that is generated (in the case of active immunization in one embodiment) or administered (in the case of passive immunization in other embodiments). In one embodiment, Intravenous Immune Globulins (IVIG) are used in patients with primary immune deficiency. These patients are born with hypo- or agammaglobulinemia and are at great risk for life-threatening infection. The life-long monthly administration of IVIG, however, affords these patients a high level of protection against bacterial and viral infections and permits them to live a normal life by providing them, passively, with a broad array of antibody specificities present in a large number of plasmapheresis donors from which the IVIG was manufactured. In one embodiment, the Avian Influenza Immune Globulin (hereinafter “AvIg”) described herein will supply critical anti-Avian Influenza antibodies, fragments thereof or combinations thereof to subjects who are at risk for this infection, or in another embodiment said anti-Avian Influenza antibodies, fragments thereof or combinations thereof will be administered to patients who are already ill as a result of this infection.

In one embodiment, the compositions and methods of the invention requires the collection of plasma from subjects who have been exposed to the Avian Influenza virus, fragments thereof, its antigen(s), or combinations thereof and the use of said plasma as a therapeutic agent, or further processing of said plasma into therapeutic materials such as immunoglobulins or hyperimmune immunoglobulin preparations, in another embodiment. In one embodiment, the immunoglobulins used in the methods and compositions of the invention, are IgG, IgM or a combination thereof.

In one embodiment, the term “antibody” includes complete antibodies (e.g., bivalent IgG, pentavalent IgM) or fragments of antibodies which contain an antigen binding site in other embodiments. Such fragments include in one embodiment Fab, F(ab′)₂, Fv and single chain Fv (scFv) fragments. In one embodiment, such fragments may or may not include antibody constant domains. In another embodiment, Fab's lack constant domains which are required for Complement fixation. ScFvs are composed of an antibody variable light chain (V_{L}) linked to a variable heavy chain (V_{H}) by a flexible hinge. ScFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

Antibodies of the invention can be monoclonal antibodies (mAb) in one embodiment, or polyclonal antibodies in another embodiment. Antibodies of the invention which are useful for the compositions, methods and kits of the invention can be from any source, and in addition may be chimeric. In one embodiment, sources of antibodies can be from a mouse, or a rat, a plant, or a human in other embodiments. Antibodies of the invention which are useful for the compositions, and methods of the invention have reduced antigenicity in humans (to reduce or eliminate the risk of formation of anti-human andtibodies), and in another embodiment, are not antigenic in humans. Chimeric antibodies for use the invention contain in one embodiment, human amino acid sequences and include humanized antibodies which are non-human antibodies substituted with sequences of human origin to reduce or eliminate immunogenicity, but which retain the antigen binding characteristics of the non-human antibody.

In one embodiment, the antibody, a fragment thereof, or combinations thereof have sufficiently high affinity and avidity to their target (Target), which may be a protein, a peptide, a nucleic acid, a sugar or a combination thereof. In one embodiment the target may be the Avian Influenza virus, or fragments of the Avian Influenza virus, or a combination thereof.

In another embodiment, fractionating the plasma sample, the sample with the immunoglobulins fragments thereof, Avian Influenza antibodies, or combinations thereof, comprises amplifying the target gene encoding for immunoglobulins fragments thereof, Avian Influenza antibodies, or combinations thereof. In one embodiment, the terms “amplification” or “to amplify” refer to one or more methods known in the art for copying a target nucleic acid, thereby increasing the number of copies of a selected nucleic acid sequence. Amplification may be exponential in one embodiment, or linear in another. In one embodiment, a target nucleic acid may be either DNA or RNA. The sequences amplified in this manner form an “amplicon.” While the exemplary embodiments described herein relate to amplification using the polymerase chain reaction (“PCR”), numerous other methods are known in the art for amplification of nucleic acids (e.g., isothermal methods, rolling circle methods, etc.) and are considered within the scope of the present invention. The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 June 1;29(11):E54-E54; Hafner et al., Biotechniques 2001 April;30(4):852-6, 858, 860 passim; Zhong et al., Biotechniques 2001 April;30(4):852-6, 858, 860.

In another embodiment, real time PCR is used in the methods of the invention. The term “real time PCR” refers in one embodiment to the process where a signal emitted from the PCR assay is monitored during the reaction as an indicator of amplicon production during each PCR amplification cycle (i.e., in “real time”), as opposed to conventional PCR methods, in which an assay signal is detected at the endpoint of the PCR reaction. Real time PCR is based in one embodiment on the detection and quantitation of a influenzaorescent reporter. The signal increases in direct proportion to the amount of PCR product in a reaction. By recording the amount of influenzaorescence emission at each cycle, it is possible to monitor the PCR reaction during exponential phase where the first significant increase in the amount of PCR product correlates to the initial amount of target template. For a general description of “real time PCR” see Dehe et al. J. Virol. Meth. 102:37-51 (2002); and Aldea et al. J. Clin. Microbiol. 40:1060-1062 (2002) (referring to the “LightCycler,” where real-time, kinetic quantification allows measurements to be made during the log-linear phase of a PCR).

The prevalence of antibodies to Avian Influenza varies considerably among different populations. Plasma will be collected in one embodiment from healthy subjects who have been previously exposed to Avian Influenza, either naturally in one embodiment, or by deliberate vaccination (immunization) in another embodiment, and who have antibodies to the virus in their plasma. These subjects are ascertained in one embodiment from populations where Avian Influenza infection is high, who have a history of a Avian Influenza infection in the past, who are found to have antibodies to Avian Influenza thorough an antibody screening program, who have antibodies as the result of deliberate immunization with Avian Influenza or with antigens associated with Avian Influenza, or a combination thereof.

The processing of subjects (“donors”) shall conform to the regulatory requirements that are applicable in the jurisdiction(s) in which the collections take place. This includes soliciting a medical history and measuring pre-donation parameters (such as blood pressure, temperature, hemoglobin, etc.). In another embodiment, after each donation the collected plasma is screened for markers for transmissible disease (e.g. anti-HIV, anti-HCV, HBsAg, Syphilis, etc.) that are applicable in the jurisdiction(s) in which the collections take place, to minimize the hazard of disease transmission. In one embodiment, all donors are screened for the presence of antibodies to Avian Influenza and, and in another embodiment, the quantity of antibodies is ascertained.

In one embodiment, the plasma used in the methods and compositions of the invention will be collected from a subject by either plasmapheresis (as source plasma) or after separation from whole blood donations (as recovered plasma). In one embodiment, “plasmapheresis” refers to a process in which the influenzaid part of the blood, is removed from blood cells by a cell separator. The separator works by either spinning the blood at high speed to separate the cells from the influenzaid, or by passing the blood through a membrane with a cellular sieve, so that only the influenzaid part of the blood can pass through. The cells are returned in one embodiment to the person undergoing treatment, while the plasma, which contains the antibodies, is collected.

In one embodiment, the term “recovered plasma” refers to the plasma that is, or has been, separated from whole blood donations. In another embodiment, “recovered plasma” refers to the process whereby heparinized blood is passed through the first filter of a cascade consisting of several filters into a stream containing the corpuscular components and a plasma stream, subjecting the plasma stream to a purification process, recombining the purified plasma and the stream containing the corpuscular particles and reinfusing the recombined blood into the subject. In one embodiment, the purified plasma is recovered, and Avian Influenza IgG, IgM, antibodies, their fragments or Avian Influenza antigens are removed prior to the recombination of the plasma and the stream containing the corpuscular particles.

After collection, the plasma is frozen in one embodiment, or stored in the liquid state for an appropriate period of time in another embodiment. Conditions of storage will be determined on the basis of optimal preservation of the anti-Avian Influenza antibodies as well as preventing contamination of the plasma. In one embodiment, usual (frozen) storage and shipping conditions that are applicable to other plasma products are employed for the Avian Influenza antibody plasma preparation.

In one embodiment, the compositions of the invention are used in the methods of the invention described herein. In one embodiment, the invention provides a method of preventing or treating Avian Influenza in a subject, comprising administering to said subject a composition comprising immunoglobulins, fragments thereof, Avian Influenza antibodies, or combinations thereof, wherein said immunoglobulins, fragments thereof, Avian Influenza antibodies, or combinations thereof are obtained from plasma of a subject immune to said Avian Influenza.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

As used herein, “subject” refers in one embodiment, to a human or any other animal which has been exposed to and is now immune to Avian Influenza. A subject refers to a human presenting to a medical provider for diagnosis or treatment of a disease, such as Avian Influenza in another embodiment. A human includes pre- and postnatal forms. In one embodiment, subjects are humans being treated for symptoms associated with Avian Influenza or a volunteer for hyperimmune antibody production following the volunteer's exposure to an attenuated virus or the like.

In one embodiment, a concentrated hyperimmune globulin appropriate for use in the treatment or prevention of Avian Influenza infection will be prepared from the collected plasma. In another embodiment, the plasma will be pooled in appropriately-sized batches and subjected to a plasma fractionation procedure which will isolate in one embodiment, and/or purify the immunoglobulin fraction and/or Avian Influenza antibodies from the plasma in other embodiments. This is done in one embodiment by the classical Cohn alcohol precipitation method, or a variant thereof, an ion exchange chromatographic method, an affinity chromatographic method, or any other suitable method such as MS-MS (tandem mass spectrometry), LC-MS (preparatory liquid chromatography and mass spectrometry), crystallization or immunopercipitation methods etc. in other embodiments. The final material will be concentrated and the titer or quantity of antibody to Avian Influenza.adjusted as appropriate. The final material will be sterile and will meet regulatory requirements as applicable in the jurisdiction of manufacture and/or use.

According to this aspect of the invention and in one embodiment, the invention provides a method of producing a pharmaceutical preparation for the prevention or treatment of an Avian Influenza, comprising: obtaining plasma from a subject immune to the Avian Influenza; pooling said plasma; fractionating said plasma wherein said fractionation isolates or purifies an immunoglobulin, a fragments thereof, an Avian Influenza antibody, or a combination thereof from the plasma; and concentrating said immunoglobulin, fragments thereof, Avian Influenza antibody, or combinations thereof.

In one embodiment, the final material may have a protein concentration of 0.5%-15%. In one embodiment, the protein concentration is between 0.1 and about 1% (w/w) or between about 1 and about 5% (w/w) in another embodiment, or between about 5 and about 10% (w/w) in another embodiment, or between about 10 and about 15% (w/w) in another embodiment. The final formulation may be appropriate for either intravenous, intrapulmonary, intracavitary or intramuscular administration, or both. Shelf life of the materials is ascertained in one embodiment, through appropriate stability studies.

In one embodiment, the pharmaceutical preparation of the invention, used in the methods of the invention comprise a carrier, excipient, flow agent, processing aid, a diluent, or a combination thereof.

In one embodiment, the compositions used in the invention further comprise a carrier, or excipient, lubricant, flow aid, processing aid or diluent in other embodiments, wherein the carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal opthalmic and oral. In one embodiment the pharmaceutical composition is administered parenterally, transmucosally, transdermally, intramuscularly, intravenously, intradermally, subcutaneously, intraperitonealy, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

The term “therapeutically effective amount” or “effective amount” refers in one embodiment, to an amount of a monovalent or combination vaccine sufficient to elicit a protective immune response in the subject to which it is administered. The immune response may comprise, without limitation, induction of cellular and/or humoral immunity. The amount of a vaccine that is therapeutically effective may vary depending on the particular antibody used in the vaccine, the age and condition of the subject, and/or the degree of infection, and can be determined by an attending physician.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexion with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, virosomes, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral, as well as self administration devices.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more. rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

The dosage regimen for treating a condition with the compositions of this invention is selected in one embodiment, in accordance with a variety of factors, such as the type, age, weight, ethnicity, sex and medical condition of the subject, the severity of the condition treated, the route of administration, and the particular compound employed, and thus may vary widely while still be in the scope of the invention.

In one embodiment, in addition to the immunoglobulins fragments thereof, Avian Influenza antibodies, or combinations thereof, used in the pharmaceutical preparations of the invention, which in another embodiment are used in the methods of the invention, the pharmaceutical preparations comprise a vaccine comprising nucleic acids encoding hemagglutinin from the index human influenza isolate A/HK/156/97.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

### AvIg Will be Used to Prevent and Treat Avian Influenza.infection in a Variety of Clinico-Epidemiological Settings

The dose of drug required is determined by the severity of the risk of developing avian influenza (the type of exposure) and the body weight of the individual. Prophylactic administration is given via the intramuscular route; intravenous administration is given in therapeutic applications in subjects who have already had symptoms attributable to avian influenza and where large doses of drug and a rapid effect are sought. These circumstances are summarized in table I.

TABLE I
Summary of clinical circumstances wherein AvIg will
be given and by what route of administration

| | Route of |
|---|---|
| Indication for AvIg Administration | Administration |
| Avian influenza outbreak | I.V. or I.M. |
| Exposure to infected or suspected infected animals | I.V. or I.M. |
| Subjects with symptoms of avian influenza, or | I.V. or I.M. |
| documented avian influenza | |
| Individuals who are at high risk to be exposed to | I.V. or I.M. |
| avian influenza (e.g. workers with chickens, | |
| workers in an aviary) | |
| Household and other close contacts of subjects with | I.V. or I.M. |
| avain influenza | |

AvIg is administered prophylactically to individuals who have been exposed to the Avian Influenza pathogenic virus. These include all individuals in or travelling to an endemic area, individuals who have been exposed to actually infected or suspected infected animals, individuals who have been exposed to subjects ill with the Avian Influenza virus and to individuals whose occupation puts them in contact with infected animals or humans. These individuals get AvIg by the intramuscular route (IM), although intravenous administration is also acceptable. Individuals who are ill with Avian Influenza, or suspected of being so, receive therapeutic doses of AvIg which are likely to be greater than prophylactic doses.

### Example 2

### Isolation and Manufacture

Source Material:

AvIg is manufactured from human plasma collected by automated plasmapheresis. and is termed source plasma or hyperimmune source plasma. In this procedure, the donor is connected to a special plasmapheresis machine for approximately 45 minutes, which automatically removes whole blood from the donor, separates the cellular elements from the liquid plasma, returns the cellular elements to the donor while retaining the plasma.

Suitable healthy donors are ascertained by a standard donor health screening questionnaire; by screening their sera or plasma for the presence of antibodies to Avian Influenza.and by measurement of the titer or quantity of antibodies present. Antibodies are acquired by two methods: first, through natural exposure to Avian Influenza virus (with our without overt symptoms) or second, by deliberate immunization with attenuated Avian Influenza virus, antigenic fragments thereof or their combinations. In certain cases an immune system booster shal be co-administered as well.

Individuals who do not have detectable antibody in their plasma/serum are offered to receive active immunization to Avian Influenza (Avian Influenza vaccine). After immunization, their antibody levels is measured, and once suitable antibody titers are developed, these individuals undergo plasmapheresis in quantities and frequencies according to local protocols and regulations. This includes collecting about 800-850 mL of plasma per procedure two times per week. Immediately after collection, the plasma is frozen and stored at no more than −18° C. until further processing and purification. All collected plasma is tested for all the appropriate communicable disease markers as required by regulatory agencies.

Manufacturing

Cohn Fractionation

Cohn plasma fractionation is used for the manufacture of a variety of plasma derivatives including a variety of normal immunoglobulin preparations (e.g. Immune Serum Globulin, Intravenous immune Globulin), immune globulin preparations (e.g. Rabies Immune Globulin, Rh Immune globulin and many others) as well as other purified proteins such as Albumin (Human), anti-hemophilic factor (factor VIII) and others.

For the manufacture of AvIg, Cohn fractions II+III are generated by alcohol precipitation and are then further purified yielding an immunoglobulin product with an IgG content of greater than 90%. The final product is formulated at an appropriate pH—at or near 7.0-7.4 for the I.M. preparation; lower pH for the I.V. preparation and adjusted to the appropriate titer. Stabilizers may be added to improve shelf life. The product is presented in solution, but lyophilization might be used as well.

Preparatory Chromatography

In preparatory chromatography, either ion exchange chromatography or affinity chromatography or a combination of the two are used. Ion exchange chromatography is used for the manfacture of various hyperimmune globulin products such as Rabies Immune Globulin or Rh Immune Globulin.

The final product using chromatographic methods has an IgG content of greater than 90%. The final product is formulated at an appropriate pH—at or near 7.0-7.4 for the I.M. preparation; lower for the I.V. preparation and adjusted to the appropriate titer. Stabilizers are added to improve shelf life. The product is presented in solution, or in a lyophilized form.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A composition for preventing an Avian Influenza in a subject, comprising immunoglobulins, fragments thereof, Avian Influenza antibodies, or combinations thereof, wherein said immunoglobulins, fragments thereof, Avian Influenza antibodies, or combinations thereof, are obtained from a plasma of a subject immune to said Avian Influenza.

2. The composition of claim 1, wherein said immunoglobulins are IgG, IgM or combinations thereof.

3. The composition of claim 1, wherein said immunoglobulin antibodies are monoclonal antibodies.

4. The composition of claim 1, wherein said immunoglobulin antibodies are polyclonal antibodies.

5. The composition of claim 1, wherein said immunoglobulin fragments are F(ab′)₂ fragments.

6. The composition of claim 1, wherein the plasma is collected from healthy subject who have been previously exposed to Avian Influenza, naturally or by deliberate vaccination (immunization), and who have IgG or IgM antibodies to the Avian Influenza virus in their plasma.

7. The composition of claim 1, wherein said plasma is collected from a subject or pool of subjects where Avian Influenza infection rate is high.

8. The composition of claim 1, wherein said plasma is collected from a subject or pool of subjects who have a history of Avian Influenza infection in the past.

9. The composition of claim 1, wherein said plasma is collected from a subject or pool of subjects who are found to have IgG or IgM antibodies to Avian Influenza through an antibody screening program.

10. The composition of claim 1, wherein said plasma is collected from a subject or pool of subjects who have antibodies as the result of deliberate immunization with Avian Influenza or with antigens associated with Avian Influenza.

11. The composition of claim 1, wherein said plasma is collected by either plasmapheresis (as source plasma) or after separation from whole blood donations (as recovered plasma).

12. The composition of claim 1, further comprising an additional therapeutic agent., adjuvant, vaccine or their combination.

13. The composition of claim 12, wherein the vaccine is an attenuated virus, an attenuated bacteria, their antigenic component or a combination thereof.

14. The composition of claim 12, wherein the adjuvant comprises mineral salts, surface-active agents and microparticles, virosomes, saponins? proteosomes, immune stimulating complexes, cochleates, quarterinary amines, pyridine, vitamin A, vitamin E; bacterial products, trehalose dimycolate, CpG oligodeoxnucleotides; cytokines, hormones, granulocyte-macrophage colony stimulating factor, dehydroepiandrosterone, 1,25-dihydroxy vitamin D3; polyanions, carriers, heat shock proteins; oil-in-water emulsions, or Freund's complete and incomplete adjuvants and their combination.

15. A method of preventing or treating Avian Influenza in a subject, comprising administering to said subject a composition comprising immunoglobulins or their fragments, Avian Influenza antibodies, or combinations thereof, wherein said immunoglobulins, fragments thereof, Avian Influenza antibodies, or combinations thereof, are obtained from plasma of a subject immune to said Avian Influenza.

16. A method of producing a pharmaceutical preparation for the prevention or treatment of an Avian Influenza, comprising: obtaining plasma from a subject immune to the Avian Influenza; pooling said plasma; fractionating said plasma wherein said fractionation isolates or purifies an immunoglobulin, or its fragment, an Avian Influenza antibody, or a combination thereof from the plasma; and concentrating said immunoglobulin, or its fragment, an Avian Influenza antibody, or a combination thereof.

17. The method of claim 16, wherein concentrating said immunoglobulin, fragments thereof, Avian Influenza antibody, or combinations thereof results in protein concentration of between about of 0.5% to about 15% (w/w).

18. The method of claim 16, wherein fractionating is done using Cohn alcohol precipitation method, a variant thereof, an ion exchange chromatographic method, an affinity chromatographic method, preparatory HPLC, LC-MS, MS-MS, immunopercipitation or similar separation methods.

19. The method of claim 16, wherein said pharmaceutical preparation comprises a carrier, excipient, flow agent, processing aid, a diluent, or a combination thereof.

20. The method of claim 19, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

21. The method of claim 19, comprising a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

22. The method of claim 16, wherein said pharmaceutical preparation is in the form of a pellet, a tablet, a capsule, a solution, a suspension, a dispersion, an emulsion, an elixir, a gel, an ointment, a cream, or a suppository.

23. The method of claim 16, wherein said pharmaceutical preparation is in a form suitable for oral, intravenous, intraaorterial, intramuscular, subcutaneous, parenteral, transmucosal, transdermal, or topical administration.

24. The method of claim 16, wherein said pharmaceutical preparation is a controlled release composition.

25. The method of claim 16, wherein said pharmaceutical preparation is an immediate release composition.

26. The method of claim 16, wherein said pharmaceutical preparation is in a liquid dosage form.

27. The method of claim 16, wherein said pharmaceutical preparation is in a solid dosage form.
